(19) United States
(12) Patent Application Publication
(10) Pub. No.: US 2023/0233244 A1
(43) Pub. Date: Jul. 27, 2023

(54) DEVICES, SYSTEMS AND METHODS FOR MEASURING TISSUE TIGHTNESS AND PERFORMING SUBDERMAL COAGULATION TO INCREASE TISSUE TIGHTNESS

(71) Applicant: Apyx Medical Corporation, Clearwater, FL (US)

(72) Inventors: Gregory Goliszek, Oldsmar, FL (US); Shawn D. Roman, Safety Harbor, FL (US); Amin Elachchabi, Hollywood, FL (US); Fredrik Jonsson, St. Petersburg, FL (US)

(21) Appl. No.: 18/013,002

(22) PCT Filed: Jul. 21, 2021
(86) PCT No.: PCT/US2021/042541

§ 371 Date: Dec. 27, 2022

Related U.S. Application Data
(60) Provisional application No. 63/055,989, filed on Jul. 24, 2020.

(51) Int. Cl. A61B 18/04; A61B 90/00

(52) U.S. Cl. CPC A61B 18/042; A61B 90/06; A61B 2018/00291

(57) ABSTRACT

The present disclosure relates to devices, systems and methods for subdermal tissue tightening through soft tissue coagulation and for use in cosmetic surgery applications. The devices, systems and methods of the present disclosure may be used for a minimally invasive application of plasma energy to subcutaneous tissue for the purpose of tightening lax tissue. The present disclosure further provides tissue tightness measurement devices, systems and methods, which are used to determine the tightness of tissue. The measurements obtained by the tissue tightness measurement devices and/or systems are used to determine when a desired tissue tightness has been achieved during a tissue tightening procedure.

### PRIORITY

This application claims priority to U.S. Provisional Pat. Application Serial No. 63/055,989, filed Jul. 24, 2020, entitled “DEVICES, SYSTEMS AND METHODS FOR MEASURING TISSUE TIGHTNESS AND PERFORMING SUBDERMAL COAGULATION TO INCREASE TISSUE TIGHTNESS”, the contents of which are hereby incorporated by reference in its entirety.

### BACKGROUND

### Field

The present disclosure relates generally to electrosurgery and electrosurgical systems and apparatuses, and more particularly, devices, systems and methods for measuring tissue tightness and performing tissue tightening through soft tissue coagulation.

### Description of the Related Art

High frequency electrical energy has been widely used in surgery and is commonly referred to as electrosurgical energy. Tissue is cut and bodily fluids are coagulated using electrosurgical energy.

Gas plasma is an ionized gas capable of conducting electrical energy. Plasmas are used in surgical devices to conduct electrosurgical energy to a patient. The plasma conducts the energy by providing a pathway of relatively low electrical resistance. The electrosurgical energy will follow through the plasma to cut, coagulate, desiccate, or fulgurate blood or tissue of the patient. There is no physical contact required between an electrode and the tissue treated.

Electrosurgical systems that do not incorporate a source of regulated gas can ionize the ambient air between the active electrode and the patient. The plasma that is thereby created will conduct the electrosurgical energy to the patient, although the plasma arc will typically appear more spatially dispersed compared with systems that have a regulated flow of ionizable gas.

Atmospheric pressure discharge cold plasma applicators have found use in a variety of applications including surface sterilization, hemostasis, and ablation of tumors. Often, a simple surgical knife is used to excise the tissue in question, followed by the use of a cold plasma applicator for cauterization, sterilization, and hemostasis. Cold plasma beam applicators have been developed for both open and endoscopic procedures. In the latter case, it is often desirable to be able to redirect the position of the cold plasma beam tip to a specific operative site. The external incision and pathway for the endoscopic tool may be chosen to avoid major blood vessels and non-target organs and may not coincide with an optimum alignment for the target internal tissue site. A means of redirecting the cold plasma beam is essential in these situations.

The heat effects of the radiofrequency (RF) alternating current used in electrosurgery on cells and tissue have been well established. Normal body temperature is 37° C. and, with normal illness, can increase to 40° C. without permanent impact or damage to the cells of a human body. However, when the temperature of cells in tissue reaches 50° C., cell death occurs in approximately 6 minutes. When the temperature of cells in tissue reaches 60° C., cell death occurs instantaneously. Between the temperatures of 60° C. and just below 100° C., two simultaneous processes occur. The first is protein denaturation leading to coagulation which will be discussed in more detail below. The second is desiccation or dehydration as the cells lose water through the thermally damaged cellular wall. As temperatures rise above 100° C., intracellular water turns to steam and tissue cells begin to vaporize as a result of the massive intracellular expansion that occurs. Finally, at temperatures of 200° C. or more, organic molecules are broken down into a process called carbonization. This leaves behind carbon molecules that give a black and/or brown appearance to the tissue.

Understanding these heat effects of RF energy on cells and tissue can allow the predictable changes to be used to accomplish beneficial therapeutic results. Protein denaturation leading to soft tissue coagulation is one of the most versatile and widely utilized tissue effects. Protein denaturation is the process in which hydrothermal bonds (i.e., crosslinks) between protein molecules, such as collagen, are instantaneously broken and then quickly reformed as tissue cools. This process leads to the formation of uniform clumps of protein typically called coagulum through a subsequent process known as coagulation. In the process of coagulation, cellular proteins are altered but not destroyed and form protein bonds that create homogenous, gelatinous structures. The resulting tissue effect of coagulation is extremely useful and most commonly used for occluding blood vessels and causing hemostasis.

In addition to causing hemostasis, coagulation results in contraction of soft tissue. Collagen is one of the main proteins found in human skin and connective tissue. The coagulation/denaturation temperature of collagen is conventionally stated to be 66.8° C., although this can vary for different tissue types. Once denatured, collagen rapidly contracts as fibers shrink to one-third of their overall length. However, the amount of contraction is dependent upon the temperature and the duration of the treatment. The hotter the temperature the shorter amount of treatment time needed for maximal contraction. For example, collagen heated at a temperature of 65° C. must be heated for greater than 120 seconds for significant contraction to occur.

Thermal-induced contraction of collagen through the coagulation of soft tissue is used in ophthalmology, orthopedic applications, and the treatment of varicose veins. The reported range of temperatures causing collagen contraction varies from 60° C. to 80° C. Therefore, once tissue is heated to within this range, protein denaturation and collagen contraction occur resulting in the reduction in volume and surface area of the heated tissue. Noninvasive radiofrequency devices, lasers, and plasma devices have been used for the reduction of facial wrinkles and rhytides caused by thermal-induced collagen/tissue contraction.

Although tissue heating devices may be effective in achieving soft tissue contraction, the evidence has been primarily limited to volume or surface area changes as measured through photographic analysis of before and after images. Measurement of the changes in biomechanical properties of the tissue resulting from soft tissue coagulation and contraction has proven more difficult. Therefore, a need exists for devices, systems, and methods capable of producing accurate measurements of biomechanical properties of tissue.

### SUMMARY

The present disclosure relates to devices, systems and methods for tissue tightening, both the skin surface and subdermal tissue planes of interest, through soft tissue coagulation for use in cosmetic surgery applications. The devices, systems and methods of the present disclosure may be used for a minimally invasive application of plasma energy to subcutaneous tissue for the purpose of tightening lax tissue. The present disclosure provides tissue tightness measurement devices, which are used to determine the tightness of tissue. The measurements obtained by the tissue tightness measurement devices are used to determine when a desired tissue tightness has been achieved during a tissue tightening procedure.

According to one aspect of the present disclosure, a tissue tightness measurement device includes a displacement mechanism that displaces tissue, for example, a suction cup that draws tissue into the suction cup when a vacuum is applied to the suction cup. The tissue tightness measurement device includes at least one position sensor that measures displacement of the tissue, i.e., the strain applied to the tissue. The tissue tightness measurement device further includes a load sensor that measures force or stress applied to the tissue as the tissue is being displaced. From these measurements, a force/displacement (stress/strain) curve may be generated. A single value of the tightness of the tissue may then be determined by calculating the slope of the “line of best fit” of the full force/displacement (stress/strain) curve or a portion of the load/displacement (stress/strain) curve over a truncated range of displacements. In this way, the tightness of the tissue is a measure of the force or load required to displace the tissue per unit of displacement.

In one aspect, at least one alignment device is provided to align the at least one position sensor to a desired location on tissue.

In another aspect, the tissue tightness measurement device is coupled to a data acquisition and display device that displays at least one of measurements taken, load/displacement curves and/or values of tissue tightness.

In one aspect, the tissue tightness measurement device communicates to the data acquisition and display device via hardwired or wireless means.

In a further aspect, the tissue tightness measurement device is further coupled, e.g., by a hardwired or wireless means, to an electrosurgical generator unit (ESU), where the ESU uses data provided from the tissue tightness measurement device to control a tissue tightening procedure and/or to determine the effectiveness of the tissue tightening procedure.

According to one aspect of the present disclosure, a tissue tightness measurement device is provided including a displacement mechanism that displaces tissue; at least one position sensor that measures displacement of the tissue; a load sensor that measures force applied to the tissue as the tissue is being displaced, the load sensor being coupled to the displacement mechanism; and a controller configured to determine a value of the tightness of the tissue based on the measured displacement of the tissue and the measured force applied to the tissue during the displacement.

In one aspect, the controller generates a force/displacement curve based on the measured displacement of the tissue and the measured force applied to the tissue during the displacement.

In another aspect, the controller determines the value of the tightness of the tissue by calculating a slope of the line of best fit over at least a portion of the force/displacement curve for a range of displacements.

In a further aspect, the displacement mechanism is a suction cup that draws tissue into the suction cup when a vacuum is applied to the suction cup.

In one aspect, a switch disposed in the suction cup and coupled to the controller, wherein when tissue is drawn into the suction cup and makes contact with the switch, the switch sends a signal to the controller to initiate acquisition of measurements.

In a further aspect, when tissue overcomes the vacuum and disengages from the switch, the switch sends a signal to the controller to stop acquisition of measurements.

In another aspect, the displacement mechanism is an interface including suture loops placed through the tissue and coupled to the load sensor.

In yet another aspect, the displacement mechanism is an interface including an adhesive pad that is coupled to the tissue and to the load sensor.

In one aspect, the at least one position sensor includes a transmitter and receiver to determine a distance from the at least one position sensor to a surface of the tissue.

In another aspect, at least two position sensors are employed to compensate for non-uniform tissue.

In a further aspect, least two position sensors are employed to compensate for tilting of the device by the user during measurements.

In still another aspect, the tissue tightness measurement device further includes at least one alignment device to align the at least one position sensor to a desired location on tissue.

In one aspect, two alignment devices are positioned on opposite sides of each of the at least one position sensor.

In another aspect, the tissue tightness measurement device further includes a transceiver that sends and receives data and/or signals with an external device.

In a further aspect, the tissue tightness measurement device further includes a housing, wherein the at least one position sensor is adjustably disposed on a surface of the housing.

In one aspect, the housing is generally cylindrical and the at least one position sensor is radially adjustable to avoid taking measurements on tenting tissue.

In another aspect, the controller is coupled to a data acquisition and display device that displays at least one of measurements taken, load/displacement curves and/or values of tissue tightness.

In yet another aspect, the controller communicates to the data acquisition and display device via hardwired or/and wireless means.

In one aspect, the data acquisition and display device is an electrosurgical generator unit (ESU).

In a further aspect, the ESU uses data provided from the controller to control a tissue tightening procedure and/or to determine the effectiveness of the tissue tightening procedure.

According to another aspect of the present disclosure, a system is provided including an electrosurgical generator unit (ESU) that generates electrosurgical energy; an applicator configured to alter tissue, the applicator being configured to receive the electrosurgical energy from the ESU to energize an electrode disposed in the applicator and to generate plasma when an inert gas is passed over the energized electrode, wherein the plasma alters the tissue; and a tissue tightness measurement device that determines tightness of the altered tissue.

In one aspect, the tissue tightness measurement device of the system includes a displacement mechanism that displaces tissue; at least one position sensor that measures displacement of the tissue; a load sensor that measures force applied to the tissue as the tissue is being displaced, the load sensor being coupled to the displacement mechanism; and a controller configured to determine a value of the tightness of the tissue based on the measured displacement of the tissue and the measured force applied to the tissue during the displacement.

In another aspect of the system, the controller generates a force/displacement curve based on the measured displacement of the tissue and the measured force applied to the tissue during the displacement.

In a further aspect of the system, the controller determines the value of the tightness of the tissue by calculating a slope of the line of best fit over at least a portion of the force/displacement curve for a range of displacements.

In one aspect, the ESU further includes a display device that displays at least one of measurements taken, load/displacement curves and/or values of tissue tightness.

In another aspect, the controller communicates to the ESU via hardwired or/and wireless means.

In yet another aspect, the ESU uses data provided from the controller of the tissue tightness measurement device to control a tissue tightening procedure and/or to determine the effectiveness of the tissue tightening procedure.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1A is an illustration of an exemplary electrosurgical system in accordance with an embodiment of the present disclosure;

FIG. 1B is an illustration of an exemplary electrosurgical generator unit in accordance with an embodiment of the present disclosure;

FIG. 1C is a flowchart illustrating an exemplary method for tightening tissue in accordance with an embodiment of the present disclosure;

FIG. 1D is a graph including stress-strain curves for human skin in accordance with an embodiment of the present disclosure;

FIG. 1E is a flowchart illustrating an exemplary method for tightening tissue and measuring tissue tightness in accordance with an embodiment of the present disclosure;

FIG. 1F is a graph illustrating mean tissue tightness per stage of tissue tightening treatment in accordance with an embodiment of the present disclosure;

FIG. 2 is an illustration of an exemplary tissue tightness measurement system in accordance with an embodiment of the present disclosure;

FIG. 3 is a perspective view of a tissue tightness measurement device in accordance with an embodiment of the present disclosure;

FIG. 4 is an exploded view of the tissue tightness measurement device shown in FIG. 3 in accordance with an embodiment of the present disclosure;

FIG. 5 is a cross-sectional view of the tissue tightness measurement device shown in FIG. 3 in accordance with an embodiment of the present disclosure;

FIG. 6 is a side view of the tissue tightness measurement device shown in FIG. 3 in accordance with an embodiment of the present disclosure;

FIG. 7 is a rear view of the tissue tightness measurement device shown in FIG. 3 in accordance with an embodiment of the present disclosure;

FIG. 8 is a perspective, bottom view of the tissue tightness measurement device shown in FIG. 3 in accordance with an embodiment of the present disclosure;

FIG. 9 is a bottom view of the tissue tightness measurement device shown in FIG. 3 in accordance with an embodiment of the present disclosure;

FIG. 10 is a perspective, top view of the tissue tightness measurement device shown in FIG. 3 with an upper housing removed in accordance with an embodiment of the present disclosure;

FIGS. 11A and 11B illustrate a method for aligning the tissue tightness measuring device in accordance with an embodiment of the present disclosure;

FIG. 12 is a block diagram of a tissue tightness measuring device in accordance with an embodiment of the present disclosure;

FIG. 13A is a perspective view of a tissue tightness measurement device in accordance with another embodiment of the present disclosure;

FIG. 13B is a perspective, bottom view of the tissue tightness measurement device shown in FIG. 13A in accordance with an embodiment of the present disclosure; and

FIG. 13C is a bottom view of the tissue tightness measurement device shown in FIG. 13A in accordance with an embodiment of the present disclosure.

It should be understood that the drawings are for purposes of illustrating the concepts of the disclosure and are not necessarily the only possible configuration for illustrating the disclosure.

### DETAILED DESCRIPTION

Preferred embodiments of the present disclosure will be described hereinbelow with reference to the accompanying drawings. In the following description, well-known functions or constructions are not described in detail to avoid obscuring the present disclosure in unnecessary detail. In the drawings and in the description which follow, the term “proximal”, as is traditional, will refer to the end of the device, e.g., instrument, apparatus, applicator, handpiece, forceps, etc., which is closer to the user, while the term “distal” will refer to the end of the device which is further from the user. Herein, the phrase “coupled” is defined to mean directly connected to or indirectly connected with through one or more intermediate components. Such intermediate components may include both hardware and software based components.

The use of thermal-induced collagen/tissue contraction has been expanded to minimally invasive procedures. Laser-assisted lipolysis (LAL) and radiofrequencyassisted lipolysis (RFAL) devices have combined the removal of subcutaneous fat with soft tissue heating to reduce the tissue laxity that often results from fat volume removal. These devices are placed in the same subcutaneous tissue plane as a standard suction-assisted lipolysis (SAL) cannula and are used to deliver thermal energy to coagulate the subcutaneous tissue including the underside of the dermis, the fascia, and the septal connective tissue. The coagulation of the subcutaneous tissue results in collagen/tissue contraction that reduces tissue laxity.

The devices, systems and methods of the present disclosure are employed for the minimally invasive application of helium-based (or based on other inert gases) cold plasma energy to subcutaneous tissue for the purpose of tightening lax tissue. A tip of a plasma generating handpiece is placed in the subcutaneous tissue plane through the same access ports used for SAL. Activation of the plasma generating handpiece in this plane causes contraction of the collagen contained in the dermis, the fascia, and the septal connective matrix through precise heating from the plasma energy.

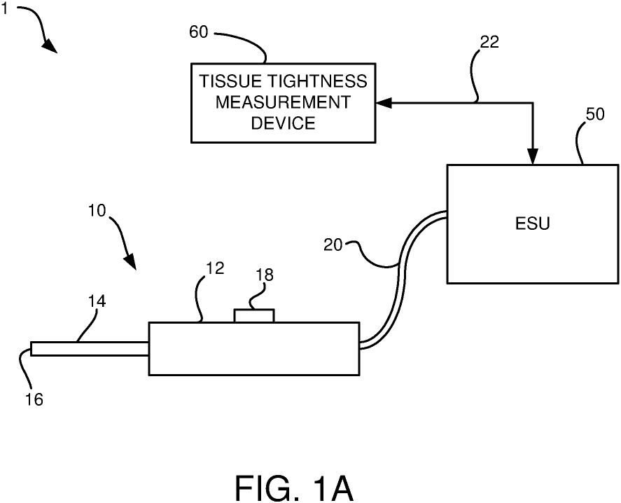

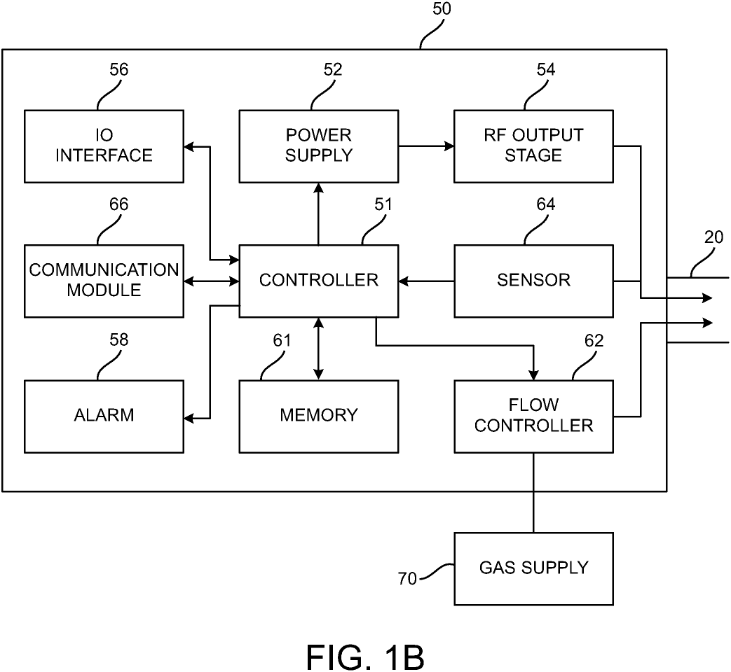

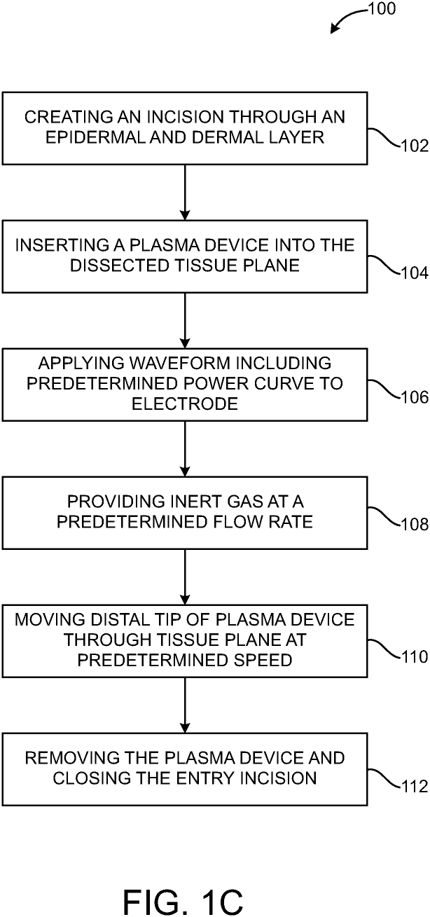

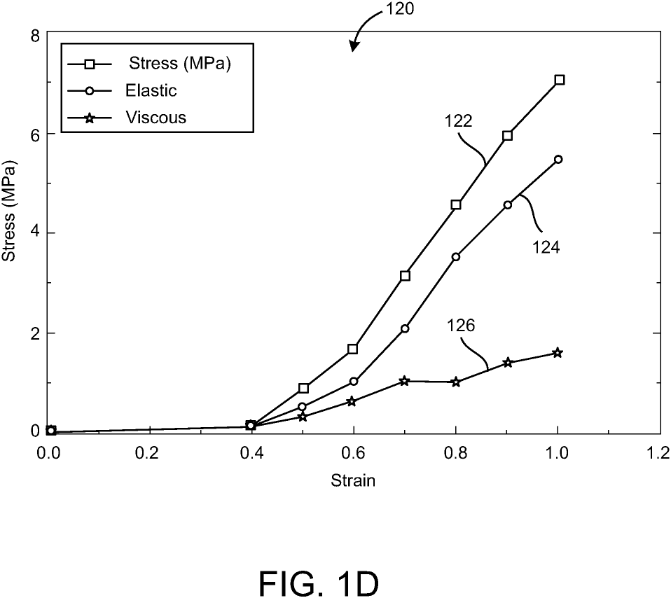

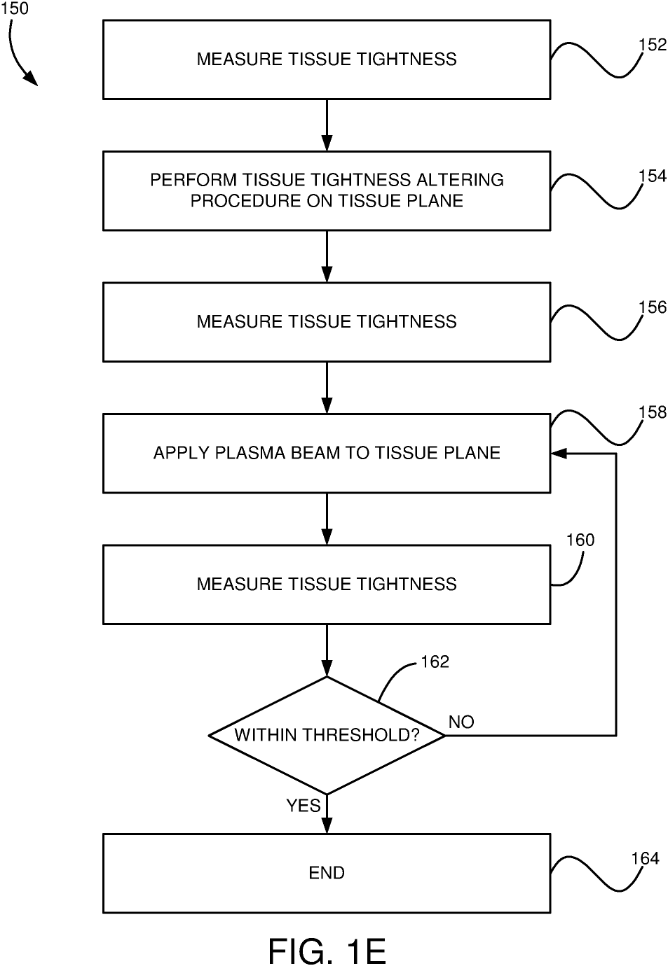

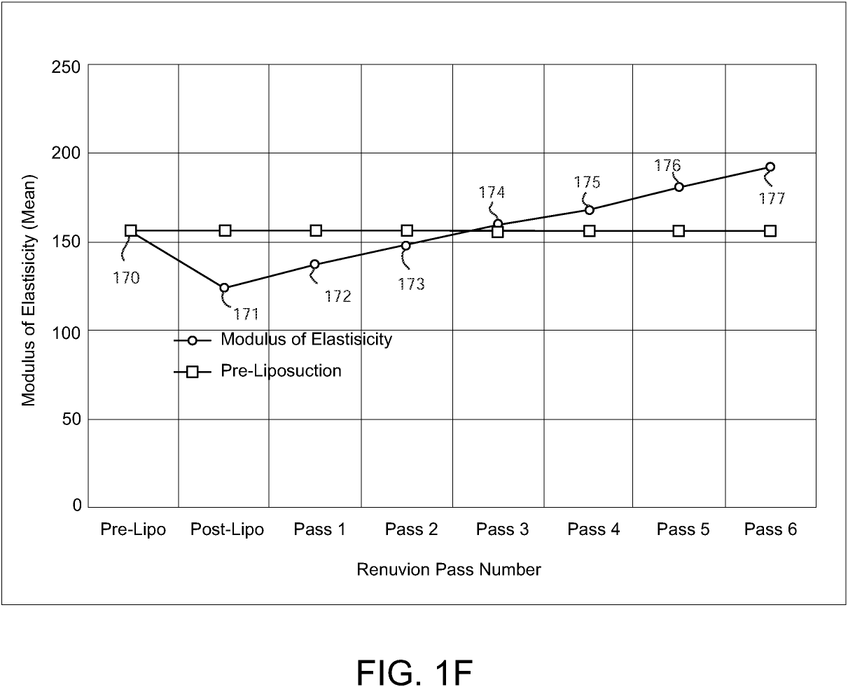

For example, referring to FIG. 1A, an electrosurgical system 1 is shown in accordance with the present disclosure. System 1 includes a plasma applicator or handpiece 10, an electrosurgical generator unit (ESU) 50, and a tissue tightness measurement device 60.

Applicator 10 is configured to receive electrosurgical energy from ESU 50 via a cable 20. Applicator 10 is further configured to receive an inert gas from a gas source. In some embodiments, the inert gas is provided from ESU 50 via cable 20. In other embodiments, the inert gas is provided via a source (not shown) separately coupled to applicator 10. Applicator 10 includes a handle housing 12 having a button 18 and a shaft 14 having a distal tip 16. When button 18 is pressed electrosurgical energy is delivered to applicator 10 by ESU 50 and inert gas is delivered to applicator 10 by the gas source. The electrosurgical energy is used to energize an electrode disposed in shaft 14, e.g., a wire electrode, a blade electrode, a needle electrode, etc. When the inert gas, is passed over the energized electrode, a plasma is generated and emitted from tip 16 to patient tissue, which allows for conduction of the RF energy from the electrode to the patient in the form of a precise plasma beam. An exemplary applicator for generating plasma is shown and described in commonly owned U.S. Pat. No. 9,060,765, the contents of which are incorporated by reference. In one embodiment, Helium is used as the inert gas because Helium can be converted to a plasma with very little energy. With the devices and systems of the present disclosure, less than 0.1% of the Helium gas employed is converted to plasma, so >99.9% of the Helium remains in a gaseous state. In some embodiments, the tissue tightness measurement device 60 may be coupled to electrosurgical generator 50 via a communication medium, via hardwired or wireless communication medium or means 22.

Referring to FIG. 1B, a block diagram of an exemplary ESU 50 is shown in accordance with an embodiment of the present disclosure. ESU 50 may include a controller or processor 51, power supply 52, radio frequency (RF) output stage 54, I/O interface 56, alarm 58, memory 61, flow controller 62, sensor 64, and a communication module 66. Controller 51 is configured to control power supply 52 to supply electrosurgical energy being output from RF output stage 54 via at least one conductor extending through cable 20 to the applicator 10. I/O interface 56 is configured to receive user input (e.g., via one or more buttons, touchscreens, etc., disposed on the housing of ESU 50) and output information (e.g., data, graphical user interfaces, etc.) received from controller 51 (e.g., via a display device, touchscreens, etc.). Audible alarm 58 is controllable via controller 51 to alert an operator to various conditions or events. Flow controller 62 is configured for controlling the flow of gas received from supply 70 to the applicator 10. The flow controller 62 is coupled to the controller 51 and receives control signals from the controller 51 based on user input via I/O interface 56 or based on an algorithm or software function stored in memory 61. Although in the embodiment shown in FIG. 1B, the flow controller 62 is disposed in the ESU 50, the flow controller 62 can be located external to the ESU 50 and disposed, for example, in a separate housing, in the applicator 10, etc.

Communication module 66 of ESU 50 is configured to communicate with other devices (e.g., tissue tightness measurement devices, client devices, servers, etc.) via a communication link (e.g., wired or wireless) to send and receive data and communications. In one embodiment, the communication module 66 may include a communication bus for receiving data from the tissue tightness measurement device 60 via communication medium 22, e.g., RS-485, USB, etc. In other embodiments, the communication module 60 may include a transceiver for wirelessly communicating with the tissue tightness measuring device 60. Although in the embodiment shown in FIG. 1B, an operator is alerted to various conditions via an audible alarm 58, in other embodiments, controller 51 may use communication module 66 to send notifications to at least one other device via the communication link (e.g., wired or wireless), where the communications are associated with the various conditions or events.

In one embodiment, sensor 64 of ESU 50 is coupled to the output of RF output stage 54. Sensor 64 is configured to sample the voltage and/or current (or any other electrical properties) of the output of stage 54 and provide the sample voltage and/or current to controller 51. Controller 51 may use the information to determine one or more properties associated with the power provided by ESU 50 to applicator 10.

It is to be appreciated that the functions of the ESU 50 may be provided through the use of dedicated hardware as well as hardware capable of executing software in association with appropriate software. In one embodiment, some or all of the functions may be performed by at least one controller 51, such as a computer or an electronic data processor, digital signal processor or embedded micro-controller, in accordance with code, such as computer program code, software, and/or integrated circuits that are coded to perform such functions, unless indicated otherwise. When provided by a processor 51, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, read only memory (ROM) for storing software, random access memory (RAM), field programmable gate arrays (FPGA), and nonvolatile storage.

The unique heating of the devices and systems of the present disclosure makes it a useful surgical tool for the coagulation of subcutaneous soft tissue similar to the LAL and RFAL devices discussed above. As the tip 16 of the applicator 10 is drawn through the subdermal plane, heating of the tissue results in instant coagulation and contraction of the tissue followed by immediate cooling.

Some devices commercially available for subcutaneous soft tissue coagulation work on the principle of bulk tissue heating. In these devices, the energy is primarily directed into the dermis and the device is activated until a pre-set subdermal temperature in the range of 65° C. is achieved and maintained across the entire volume of tissue. As discussed above, at 65° C., the tissue being treated must be maintained at that temperature for greater than 120 seconds for maximal contraction to occur. Although these devices may be effective in achieving soft tissue contraction, the process of heating all of the tissue to the treatment temperature and maintaining that temperature for extended periods can be time consuming. In addition, during this process, the heat eventually conducts to the epidermis requiring constant monitoring of epidermal temperatures to ensure they do not exceed safe levels.

In contrast to previous approaches, handpiece 10 and ESU 50 of system 1 of the present disclosure achieve soft tissue coagulation and contraction by rapidly heating the treatment site to temperatures greater than 85° C. for between about 0.040 and about 0.120 seconds. It is to be appreciated that handpiece 10 and/or ESU 50 may include a processor configured to ensure the heat (provided via the tip of the applicator, e.g., tip 16) applied to patient is maintained for between about 0.040 and about 0.120 seconds. For example, when button 18 of applicator 10 is pressed, a processor in applicator 10 or controller/processor 51 in ESU 50 may be configured to apply electrosurgical energy to the electrode continuously for between about 0.040 and about 0.120 seconds.

In some embodiments, a temperature sensor (e.g., an optical sensor, thermocouple, resistance temperature detector (RTD)) may be included in the distal tip (e.g., tip 16) or be otherwise in communication with the applicator 10 and/or the ESU 50. The temperature sensor provides temperature readings of the target tissue to the processor. The processor 51 is configured to adjust the power outputted by ESU 50 and the time duration that the heat is applied to the target tissue to ensure that temperatures greater than 85° C. for between about 0.040 and about 0.120 seconds are reached.

In some embodiments, a predetermined power curve is stored in memory 61 and applied by ESU 50 to the electrode in applicator 10. The predetermined power curve ensures the tissue is heated to temperatures of at least 85° C. for between about 0.040 to about 0.120 seconds. Furthermore, in accordance with the present disclosure, other properties associated with the application of plasma may be controlled to guarantee the temperatures of the tissue heated. For example, the flow rate of the inert gas provided to distal tip 16 and the speed that the tip 16 is moved through the tissue plane may be selected to ensure the target temperatures described above are reached.

Referring to FIG. 1C, a method 100 of coagulating a subcutaneous layer of tissue using the handpiece or applicator 10 and ESU 50 of system 1 is shown in accordance with an embodiment of the present disclosure.

Initially, in step 102, an incision, i.e., an entry incision, is created through the epidermal and dermal layers of a patient at a location appropriate for a particular procedure. In step 104, the tip 16 of the applicator 10 is inserted into the dissected tissue plane. Next, the applicator is activated (e.g., is activated to coagulate and/or ablate tissue to create a desired effect, e.g., (i) tighten tissue, (ii) shrink tissue and/or (iii) contour or sculpt the body).

When the applicator 10 is activated, in step 106, ESU 50 applies a waveform including a predetermined power curve to the electrode of applicator 10. In one embodiment, the predetermined power curve is configured such that electrosurgical energy is provided in a pulsed manner, with each pulse having a predetermined time duration and with the electrosurgical generator outputting a predetermined output power when the waveform is applied. For example, in one embodiment, the power curve is configured such that the predetermined time duration of a pulse is between about 0.04 seconds and about 0.120 seconds and the predetermined output power is between about 24 Watts and about 32 Watts.

Furthermore, when applicator 10 is activated, in step 106, a gas source (e.g., integrated with ESU 50 or separate from ESU 50) is configured to provide inert gas to the distal tip 16 of applicator 10 at a predetermined flow rate, in step 108, to generate plasma. In one embodiment, the inert gas used is helium and the predetermined flow rate is between about 1 liter per minute and about 5 liters per minute.

In step 110, the user moves the distal tip 16 of the applicator 10 through the tissue plane at a predetermined speed while the tip 16 is emitting plasma. In one embodiment, the predetermined speed ranges from about 1 centimeter per second to about 3 centimeters per second. It is to be appreciated that, in the method 100, the predetermined power curve of the waveform, the predetermined flow rate of the inert gas, and the predetermined speed of the tip 16 through the tissue plane are selected such that, when steps 106-110 are performed, the temperature of the tissue being heated by the plasma emitted from the applicator 10 reaches at least 85° C. and the tissue is not heated in bulk (e.g., in areas surrounding or further away from the target tissue), but instead is heated instantaneously and cools quickly after treatment. After the desired effects are achieved, the applicator 10 is removed, and the entry incision is closed, in step 112.

Unlike with bulk tissue heating, the rapid heating of tissue performed by the system 1 of the present disclosure allows the tissue surrounding the treatment site to remain at much cooler temperatures resulting in rapid cooling after the application of the energy through conductive heat transfer. Additionally, the energy provided to the tissue using the electrosurgical apparatuses of the present disclosure is focused on heating the fibroseptal network (FSN) instead of the dermis. The majority of soft tissue contraction induced by the subcutaneous energy delivery devices is due to its effect on the FSN. Because of these unique heating and cooling properties of the electrosurgical apparatuses of the present disclosure, immediate soft tissue contraction can be achieved without unnecessarily heating the full thickness of the dermis.

Method 100 may be used to perform collagen/tissue contraction in minimally invasive, percutaneous tissue tightening procedures to reduce the tissue laxity that often results from fat volume removal, e.g., during liposuction. Applicator 10 may be placed in the same subcutaneous tissue plane (or any tissue plane of interest) as a liposuction cannula and used to deliver thermal energy to coagulate the subcutaneous tissue including the septal connective tissue and, to a lesser extent, the underside of the dermis and fascia. The coagulation of the subcutaneous tissue results in collagen/tissue contraction that reduces tissue laxity, e.g., skin laxity.

Elasticity is defined as the ability of an object or material to resume its normal shape after being stretched or compressed. The rubber in a balloon is an example of a material that is near 100% elastic. When a balloon that was just inflated is compressed again, it will quickly spring back to its original shape. In contrast, modeling clay is a 100% plastic material. When mechanical force is applied, the modeling clay will retain the resulting shape even after the force is removed. Human tissue, e.g., human skin, is not completely elastic or completely plastic. Tissue deforms with applied force but returns to its original position after a slight delay when the force is removed. This combined elastic and plastic behavior of human tissue is called viscoelasticity. The elastic and plastic behavior of skin, connective tissue, and other materials is most commonly graphically displayed on a stress-strain curve. The stress-strain curve is unique for a given material and is established by recording the amount of deformation or displacement of the material (i.e., strain) corresponding to a tensile or compressive load (i.e., stress).

For example, referring to FIG. 1D, a graph 120 is shown including the total (curve 122), elastic (curve 124), and viscous (curve 126) stress-strain curves established for human skin.

As can be seen on the total stress-strain curve 122 in FIG. 1D, the stress-strain behavior of skin is composed of three distinct phases. In the first phase corresponding to small deformations or displacements of the tissue (i.e., strain less than or equal to 0.4), the collagen fibers and fibrils in the tissue offer little resistance to deformation as they begin to uncrimp or unfold. In the second phase corresponding to higher deformations (i.e., strains between 0.4 and 0.6), the collagen fibers begin to offer resistance to the deformation and begin to carry the load of the force applied. The stress-strain relationship during this second phase is highly linear and is dominated by the elastic characteristics of the tissue and the collagen. In the third phase corresponding to large deformations (i.e., strains greater than 0.6), the crosslinks between the collagen fibrils begin to yield and eventually failure of the tissue occurs (such as tearing).

Analyzing the stress-strain curve of skin and connective tissue during the highly linear second phase described above can provide significant insight into its elastic characteristics. For example, calculating the slope of this linear portion of the stress-strain curve provides a measure of the tissue’s resistance to deformation by a mechanical force (i.e., slope = change in stress/change in strain). In material science and engineering disciplines, this resistance is most commonly referred to as the elastic modulus or the modulus of elasticity of a material. In reference to skin or human tissue, especially in cosmetic plastic surgery and dermatology, it is most commonly referred to as tissue firmness or tissue tightness. Changes in the firmness or tightness of tissue will result in changes to the slope of this linear portion of the stress-strain curve.

In one embodiment, a tissue tightness measurement device 60 may be used in system 1 to provide measurements of the firmness or tightness of a tissue surface affected by fat volume removal and/or the coagulation of a subcutaneous tissue plane of interest as a feedback signal to a processor in generator 50 or in applicator 10, such that a desired tissue tightness or firmness can be achieved during a tissue tightening procedure performed using applicator 10.

Referring to FIG. 1E, an exemplary method 150 for the treatment of lax soft tissue using an electrosurgical system including plasma energy device, such as system 1, is shown in accordance with an embodiment of the present disclosure. In step 152, before any treatment of patient tissue is performed, the tightness of a portion of tissue, e.g., skin of a patient, is measured using a tissue tightness measurement device 60 to establish a baseline for the tightness of the tissue. In step 154, a tissue tightness altering procedure (e.g., liposuction) is performed on a subcutaneous tissue plane, e.g., below the portion of tissue that was measured in step 152. In step 156, the tightness of the tissue is measured again using the tissue tightness measurement device 60. The measurements of steps 152 and 156 may be used to determine the change in tissue tightness resulting from the procedure performed in step 154. In step 158, a plasma beam is generated by applicator 10 (e.g., in the manner described above in relation to steps 106-110 of FIG. 1C) in a single pass or predetermined number of passes within the subcutaneous tissue plane of interest to coagulate tissue and thereby tighten the tissue.

In step 160, the tightness of the tissue is measured again and, in step 162, it is determined (e.g., by a processor in generator 50) if the tightness from step 160 is within a threshold value (or optionally within a predetermined range). In one embodiment, the threshold value is based on the tightness measurement obtained in step 152 (e.g., the tissue tightness of the patient before the procedure of step 154 is performed). If it is determined that the tightness is not within the predetermined threshold, in step 162, applicator 10 is used to apply plasma in a second pass or predetermined number of passes within the subcutaneous tissue plane of interest, in step 158, and steps 158-162 are repeated until the tightness of the tissue is within the predetermined threshold or range (or optionally, above the predetermined threshold). Alternatively, if it is determined that the tightness is within the predetermined threshold (or above the predetermined threshold), in step 162, method 164 ends as the tissue is deemed to have the desired tightness or firmness. For example, the threshold value may be the baseline value obtained in step 152; where if the value obtained in step 160 is greater than the value obtained in step 152, it is determined that the tissue has become tightened. In this example, the tissue tightness determined in step 152 is compared to the tissue tightness determined in step 160. As another example, the threshold value may be the baseline value plus a predetermined offset; where if the value measured in step 160 is greater than the baseline value plus the offset, tissue tightening has been achieved.

FIG. 1F illustrates exemplary results of the method 150 performed in accordance with the present disclosure. The values were obtained in conjunction with a liposuction treatment and subsequent passes of a plasma device of the present disclosure, e.g., plasma applicator 10. FIG. 1F represents a graph of mean tissue tightness values, i.e., mean modulus of tightness values, for each stage of treatment. For example, data point 170 was obtained by measuring tissue tightness before the liposuction treatment, as in step 152 above, to obtain the baseline; data point 171 was obtained after liposuction was performed, as in step 154; and data points 172, 173, 174, 175, 176 and 177 were obtained after each subsequent pass of the plasma device. FIG. 1F demonstrates an immediate decrease in tissue tightness following liposuction as the fat volume is removed and tissue laxity increases. With each subsequent pass of helium plasma treatment, an increasing linear trend in tissue tightness is evident. The summary statistics of the change in the tissue tightness values for each stage of treatment compared to the previous stage of treatment is presented below in Table 1:

| Stage of Treatment Comparison | Mean Difference (SD) g/in |
|---|---|
| Pre-Lipo-Post-Lipo | -32.0 (15.0) |
| Pass 1 - Post-Lipo | 13.1 (5.5) |
| Pass 2 - Pass 1 | 11.7 (4.8) |
| Pass 3 - Pass 2 | 10.6 (3.9) |
| Pass 4 - Pass 3 | 9.4 (4.3) |
| Pass 5 - Pass 4 | 12.6 (4.3) |
| Pass 6 - Pass 5 | 11.4 (7.0) |

The average decrease in tissue tightness as a result of liposuction was 32.0 g/in, i.e., grams of force/inches of distraction. So basically, it requires 32 grams of force to distract the tissue one inch. In this specific example, the amount of force it took to distract the tissue one inch decreased by 32 grams after performing liposuction. On average, a single treatment pass with the helium plasma energy device (e.g., applicator 10) increased the tightness of the tissue by about 11.5 g/in. Therefore, on average, the treated tissue began to exceed its pre-liposuction tightness value by the third treatment pass. Passes four through six served to increase the tissue tightness over and above the pre-liposuction value.

In some embodiments, the controller/processor 51 in generator 50 may be configured to provide an alert (e.g., an alarm sound via alert 58 and/or a display notification via IO interface 56) if it is determined that the tissue tightness is within/or above the predetermined threshold, in step 162. In some embodiments, the processor 51 in generator 50 may be configured to adjust the waveform applied to the electrode in applicator 10 based on the amount the tissue tightness is above the predetermined threshold, in step 162. Additionally or alternatively, in some embodiments, the processor 51 in generator 50 may adjust the waveform based on the amount the tissue tightness measured in step 156 is above the predetermined threshold. In either case, a mapping between the amount of adjustment necessary to the waveform (e.g., in intensity, frequency, duration of pulses, etc.) as a function of the difference between the currently measuring tightness (e.g., in step 156 or step 160) and the predetermined threshold may be stored in a memory 61 of generator 50 and used by the processor 51 in generator 50 to make adjustments to the waveform. It is to be appreciated that the predetermined threshold may be derived from the baseline measurement taken in step 152 or may be an adjustable value entered by an operator into the IO interface 56 of the ESU 50.

It is to be appreciated that in some embodiments, step 152 and 154 may be removed from method 150 and steps 156-164 may be performed alone without the performance of a tissue tightness altering procedure.

The present disclosure provides various tissue tightness measurement devices, which may be used in system 1 and in the method 150 described above.

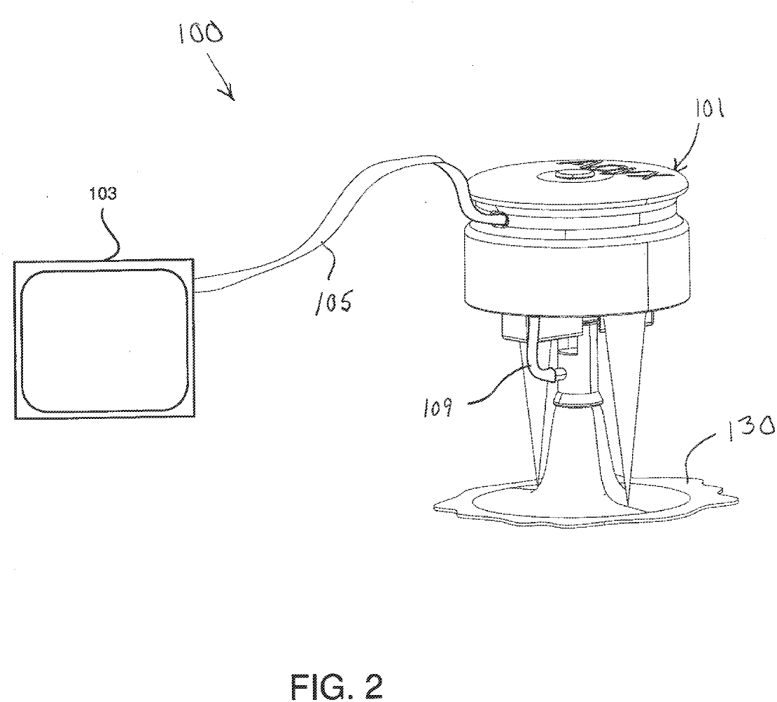

Referring to FIG. 2, a tissue tightness measuring system 100 is provided. The system 100 includes tissue tightness measuring device 101 coupled to a data acquisition unit 103 via a cable 105. The tissue tightness measuring device 101 employs at least one load sensor and at least one displacement sensor to determine tightness of tissue. It is to be appreciated that in certain embodiments, the data acquisition unit 103 may be disposed in the electrosurgical generator unit or may be the electrosurgical generator unit.

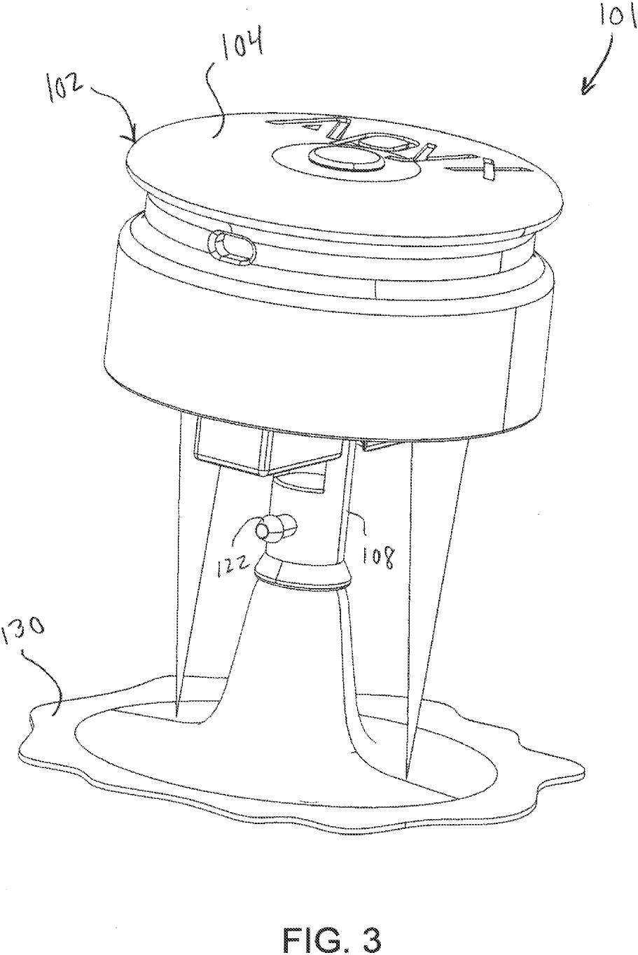

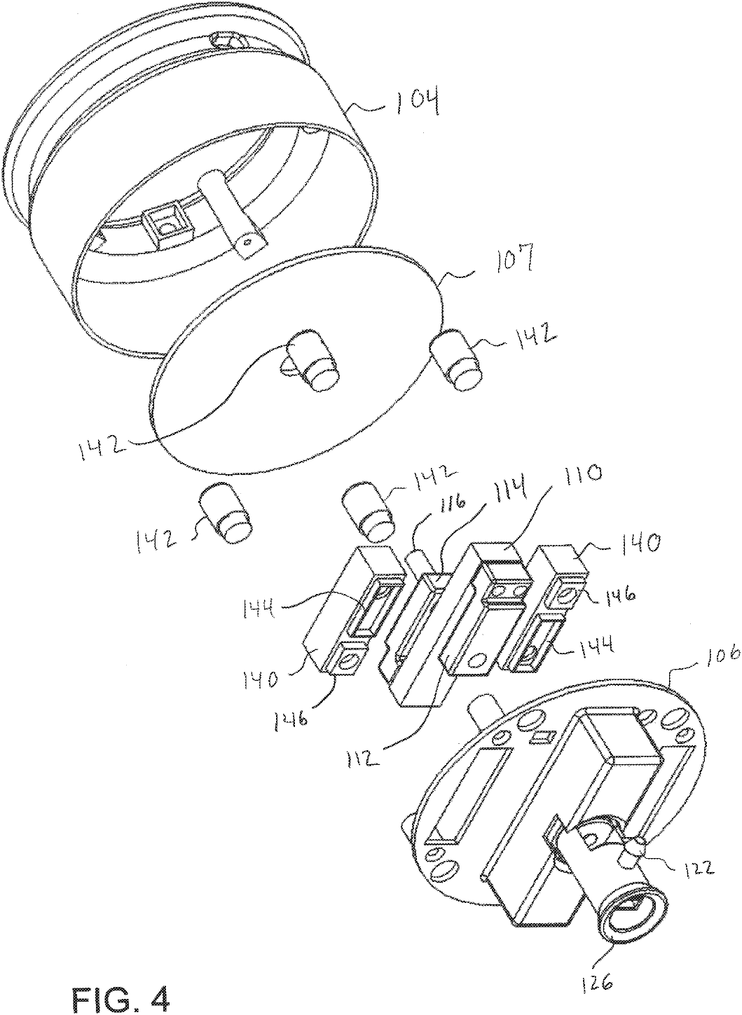

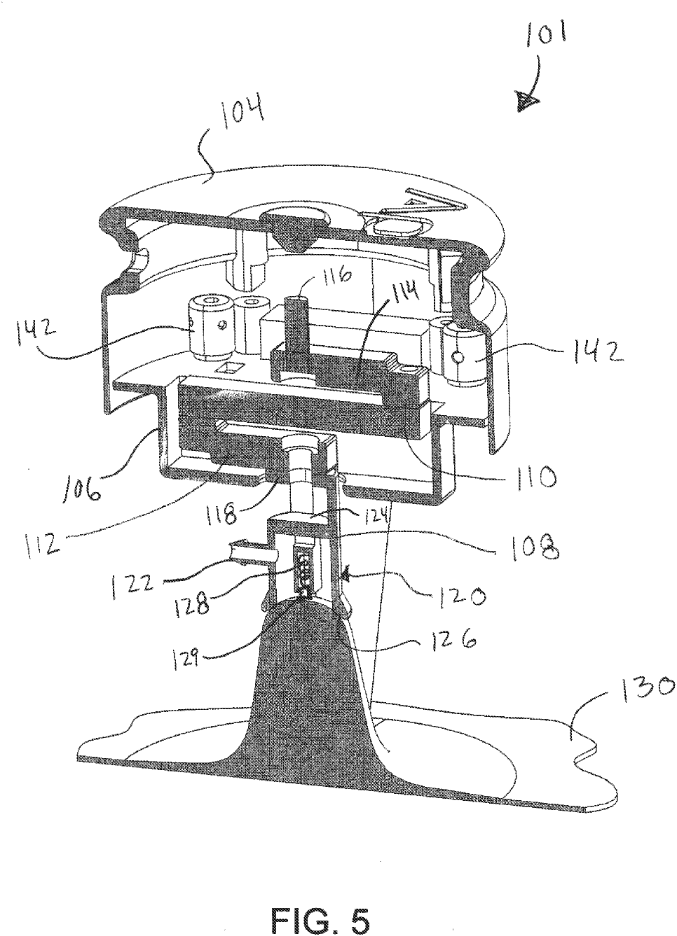

Referring to FIGS. 3-5, the device 101 includes a generally cylindrical housing 102 with a displacement mechanism, e.g., suction cup 108, extending therefrom. As can be seen more clearly in FIGS. 4 and 5, the housing 102 includes an upper housing 104 and a lower housing 106. A disc-shaped printed circuit board 107 is disposed in the housing, the details of which will be described below. Extending from the lower housing 106 is the displacement mechanism, e.g., suction cup 108, which is coupled to a load cell/sensor disposed in the housing 102.

Referring to FIG. 5, a cross sectional view of the device 100 is illustrated. A load cell/sensor 110 is disposed in a portion of the lower housing 106, wherein the printed circuit board 107 has been removed for clarity. The load cell/sensor 110 includes a lower arm 112 which is coupled to the suction cup 108 and an upper arm 114 which is coupled to the portion of the lower housing 106. The upper arm 114 further includes a shaft 116. It is to be appreciated that the shaft 116 may be employed during a calibration process of the load cell/sensor 110, where an external calibrating device is coupled to the shaft 116. In use and/or after calibrating the load cell/sensor 110, the shaft 116 may optionally be removed. In other embodiments, the shaft 116 may be entirely eliminated.

The suction cup 108 includes an upper portion 118 that is coupled to the lower arm 112 of load cell/sensor 110. The suction cup 108 further includes a cylindrical shaped lower portion 120 with a vacuum port 122 disposed along a wall of the lower portion 120. A first end 124 of the lower portion 120 is closed and a second end 126 of the lower portion 120 is open forming a circular aperture. In use, the second end 126 is placed in contact with tissue 130, e.g., skin, making an airtight seal so when a vacuum is applied to port 122, the tissue will be caused to enter, or be sucked into, the second end 126 of the lower portion 120 of suction cup 108. A switch 128 is disposed in the center of the suction cup 108 along a longitudinal axis of the cup 108 and extending toward the second end of the cup 108. The switch 128 includes a spring-loaded component 129, where when tissue is sucked into the second end 126 of cup 108 and the tissue comes in contact with the switch 128 (or spring-loaded component 129), a signal is provided to a controller/processor on PCB 107 to (i) zero the position sensors and the load cell/sensor and (ii) initiate the acquisition of data. Eventually, as the force to displace the tissue overcomes the suction force, the tissue is pulled free of the switch 128 (i.e., detaches from the suction cup 108) which sends a signal to the controller/processor of PCB 107 to stop acquisition of data at that point.

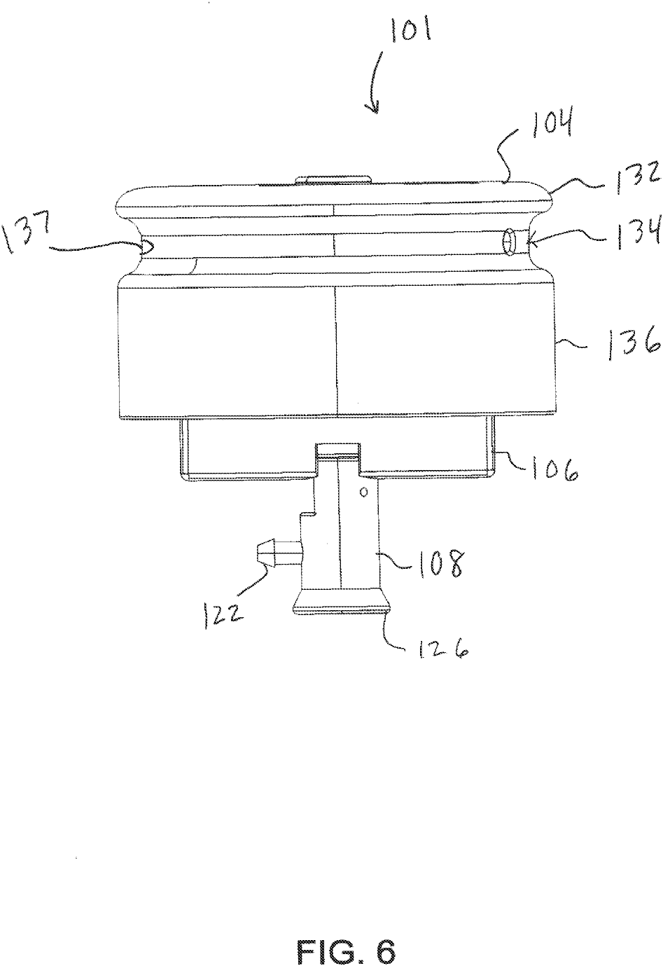

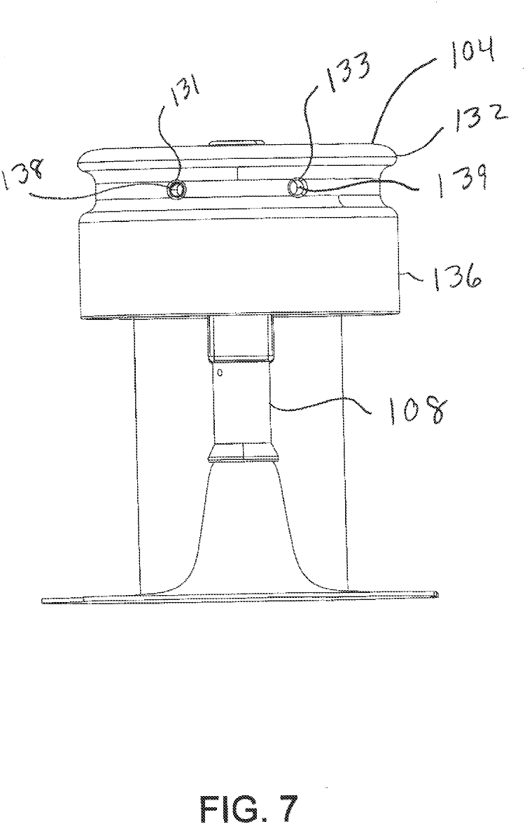

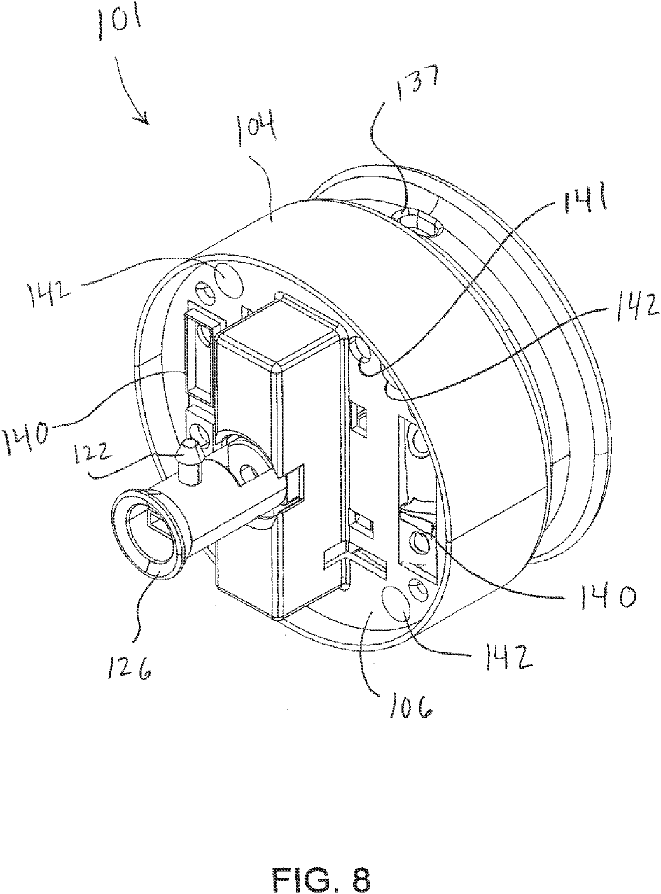

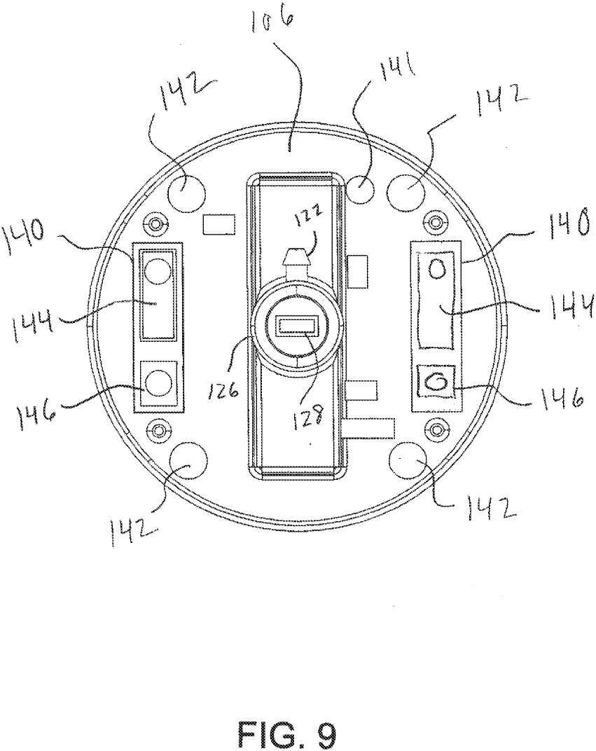

Referring to FIGS. 6 and 7, the upper housing 104 includes an upper portion 132, a recessed portion 134 and a lower portion 136. The recessed portion 134 is configured to facilitate gripping of the device 101. The recessed portion 134 includes one slot 137 and two small circular openings 138, 139. The slot 137 is an exit port for the at least one cable 105 (shown in FIG. 2). It is to be appreciated that the at least one cable 105 may include at least one electrical cable that couples the PCB 107 to a data acquisition unit 103 (or optionally, to an electrosurgical generator unit ESU) and at least one tube that goes to a vacuum unit for applying vacuum to port 122. It is further to be appreciated that the electrical cable and tubing may be combined into one combination cable/tube assembly. As shown more clearly in FIGS. 8 and 9, tube 109 (which may be partially disposed in cable assembly 105) that enters slot 137 will exit housing 102 via aperture 141 to then be coupled to port 122. Furthermore, in one embodiment, the data acquisition unit 103 and vacuum unit may be combined into one unit. In yet another embodiment, the data acquisition unit 103 and vacuum unit may be combined into an electrosurgical generator unit, e.g., ESU 50, used to generate monopolar, bipolar, and plasma energy for the purpose of cutting, coagulating, and ablating soft tissue during surgical procedures. The data acquisition unit 103 may further include an input/output interface for receiving input/instructions from a user and for displaying data to a user, e.g., measured values, stress/strain curves, etc.

The two small circular openings 138, 139 are configured to accommodate two buttons 131, 133 respectively which are electrically coupled to the PBC 107. One button, e.g., button 131, activates alignment devices (which will be described below) and the other button, e.g., button 133, is a back-up to allow manual activation of the data acquisition (as opposed to relying on the switch 128 inside the suction cup 108).

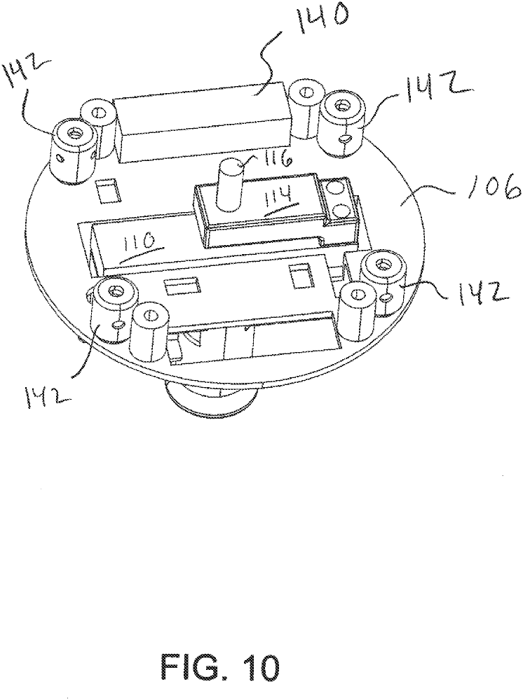

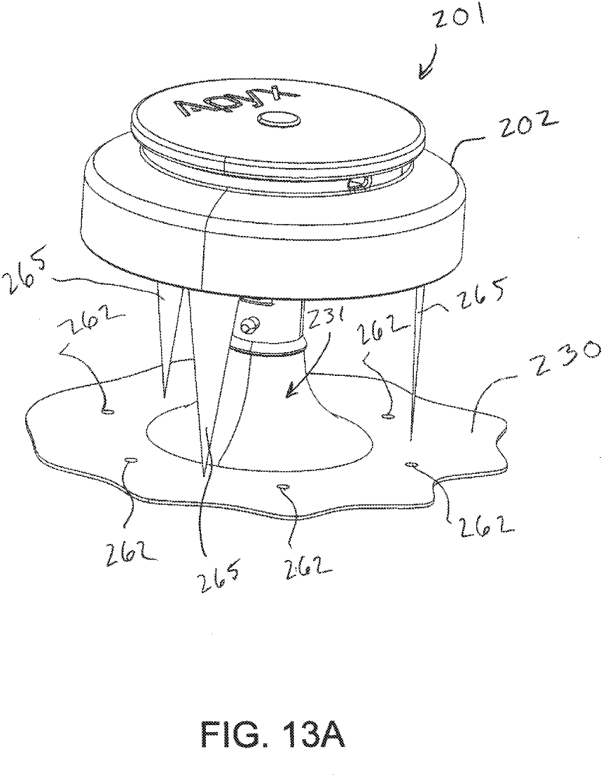

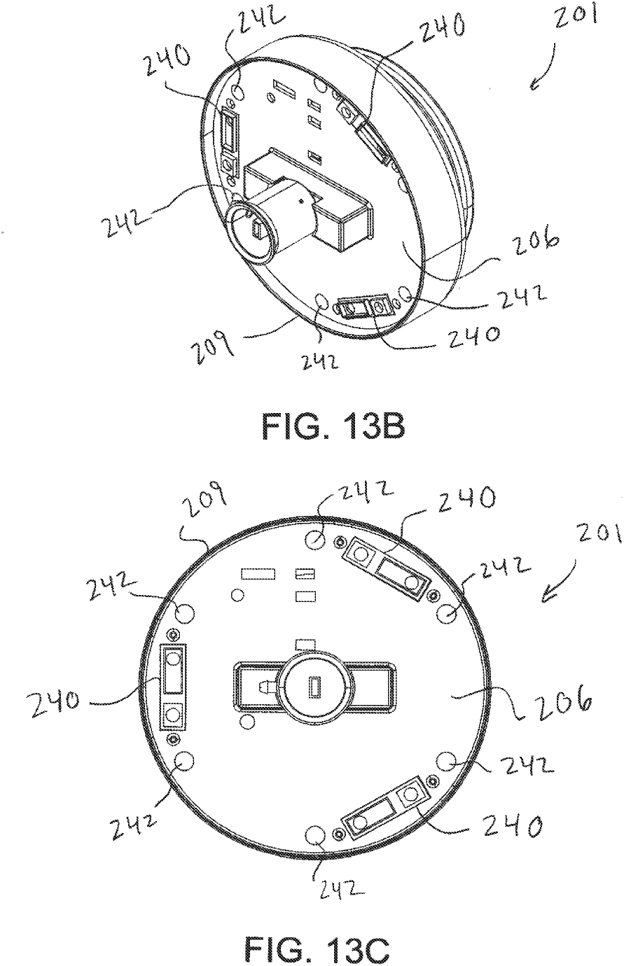

Referring to FIGS. 8-10, a perspective bottom view of device 101 is shown in FIG. 8, bottom view of the device 101 is shown in FIG. 9 and FIG. 10 illustrates a top perspective view of device 101 with the upper housing 104 removed. The device 101 includes two position or displacement sensors 140 and a plurality of alignment devices 142. Each displacement sensor 140 includes a transmitter 144 and a receiver 146 to determine a distance from the position sensor 140 to the surface of the tissue. The alignment devices 142 may include, but are not limited to, a laser and/or a light emitting diode (LED). In one embodiment, at least one alignment device 142 is associated with each position sensor 140 to align the sensor 140 to a location on the skin. For example, an alignment device 142 may be disposed near the center of the position sensor 140, e.g., approximately between the transmitter 144 and the receiver 146. In another embodiment, two alignment devices 142 are placed on each side of the displacement sensors 140 and are used to align the sensor 140 to a location on the skin. The alignment devices 142 provide visual reference to the user on the location of the displacement sensors 140. That is, the alignment devices 142 show the user where the sensors 140 are “looking” so the user can make a decision on how to best orient the unit when taking a measurement. Additionally, the user may mark the location of the dots 262 generated by the alignment devices (for example, as shown in FIG. 13A) on the tissue being measured using a tissue marker. This would provide visual reference to the user as to the position of the displacement sensors on previous measurements to ensure that all measurements are taken with the displacement sensors in the same location on the tissue to ensure consistency of the measurements.

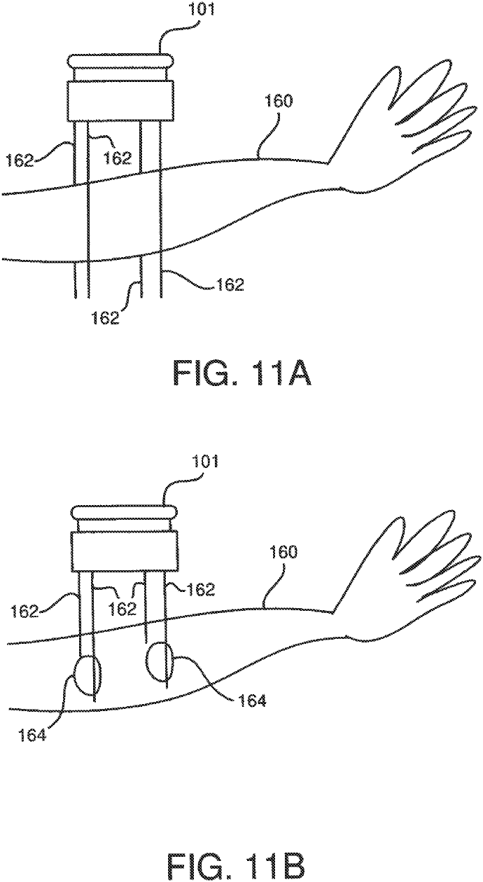

It is to be appreciated that it would be ideal if the sensors 140 were measuring off of a flat surface as a baseline. However, this may not be possible when taking measurements on the human body, but the user should attempt to choose surfaces of the body that are as consistent as possible. The alignment devices 142 provide the user with a visual reference to rotate the device 101 as necessary to maximize the consistency. For example, let’s say a user is using a two-sensor device 101 to take a measurement on the underside of a patient’s forearm. If the device 101 with the two sensors 140 is positioned so that sensors 140 or alignment devices 142 associated with the sensor 140 hang off the sides of the subject’s arm 160 or go around the radius of the arm 160 (as shown in FIG. 11A), it could impact the readings. As shown in FIG. 11A, the light beams 162 emitted by the alignment devices 142 go around the radius of the arm 160. It would be better to position the sensors 140 so the sensors 140 are in line with the subject’s forearm (i.e., one positioned toward the subject’s hand, the other positioned toward the subject’s elbow, as shown in FIG. 11B). As shown in FIG. 11B, the device 101 is rotated so the light beams 162 generated by the alignment devices 142 appear on the subject’s forearm to provide an indication of where the position sensor 140 is reading. It is to be appreciated that the area 164 between two alignment beams 162 is the area being read by the position sensor 140. This would give the sensors 140 relatively consistent surfaces to measure off of on either side of the tissue that is being pulled.

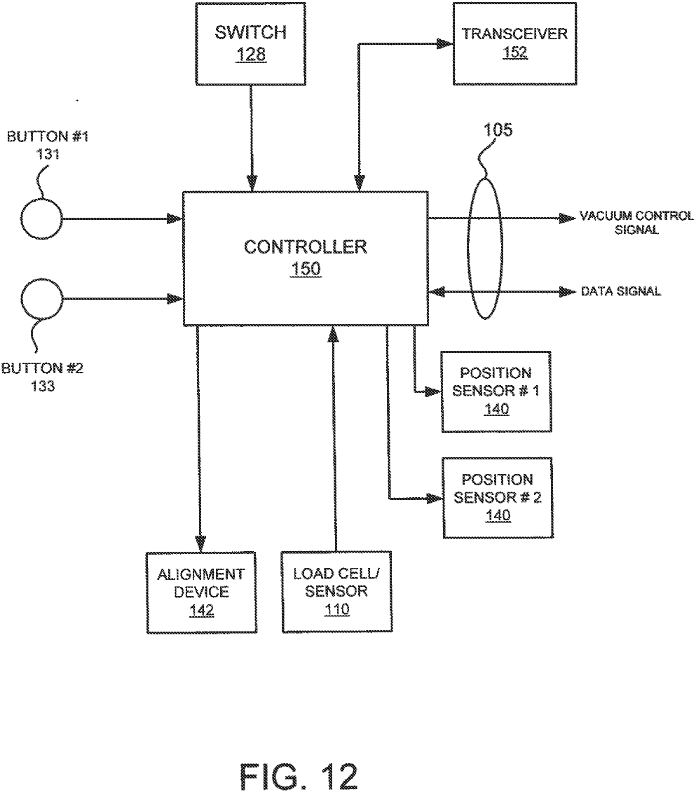

Referring to FIG. 12, a block diagram of the components of the device 101 is illustrated. A controller 150, e.g., a processing device, is provided and disposed on the PCB 107. The various components, e.g., button 1131, button 2 133, switch 128, alignment device(s) 142, load cell/sensor 110, and position sensor(s) 140, are in communication with the controller 150 by various means known in the art, e.g., wires, traces, vias, throughholes, etc. Additionally, controller 150 is in communication with data acquisition unit 103 via cable 105. In one embodiment, the controller 150 may send measurement signals, e.g., measurements obtained from position sensor(s) 140 and load cell/sensor 110, to the data acquisition unit 103 and send a control signal to activate/deactivate the vacuum unit. In another embodiment, the controller 150 may send the measurement and control signals to the data acquisition unit 103 wirelessly via a transceiver 152 coupled to the controller 150.

It is to be appreciated that the functions of the device 101 may be provided through the use of dedicated hardware as well as hardware capable of executing software in association with appropriate software. In one embodiment, some or all of the functions may be performed by the controller 150, such as an electronic data processor, digital signal processor or embedded micro-controller, in accordance with code, such as computer program code, software, and/or integrated circuits that are coded to perform such functions, unless indicated otherwise. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, read only memory (ROM) for storing software, random access memory (RAM), and nonvolatile storage.

In use, the displacement mechanism, e.g., suction cup 108, of device 101 is placed on an area of tissue of interest while the tissue is at rest, i.e., a normal or unstressed state. The device 101 is activated upon pressing button #1 131. Upon activation, a control signal is sent to the vacuum unit by the controller 150 via the at least one cable 105 or wirelessly via transceiver 152. Additionally, the alignment devices 142 and position sensors 140 are activated. Via the vacuum unit, a vacuum is applied to the suction cup 108 through the vacuum inlet or port 122 to draw the tissue, e.g., skin, into the second end 126 of the suction cup 108. The switch 128 located inside of the suction cup 108 determines when the tissue is engaged into the suction cup 108. When the tissue comes in contact with the switch 128 (or spring-loaded component 129), a signal is provided to controller 150 on PCB 107 to (i) zero the position sensors 140 and the load cell/sensor 110 and (ii) initiate the acquisition of data. As the user pulls the device 101 away from the body or patient, the displacement sensors 140 are used to determine the distance of the lower surface of the lower housing 106 from the tissue. At substantially the same time, the load cell/sensor 110 is measuring the force on the lower arm 112. Eventually, as the force to displace the tissue overcomes the suction force, the tissue is pulled free of the switch 128 (i.e., detaches from the suction cup 108) which sends a signal to the controller 150 to stop acquisition of data at that point. Once the data acquisition has ceased, the controller 150 may instruct the vacuum unit to stop and turn off the alignment devices 142.

Once the tissue disengages from the switch 128/suction cup 108, a load /displacement (stress/strain) curve may be generated, for example, by software executing in the data acquisition unit 103. A single value of the tightness of the tissue may then be determined by calculating the slope of the “line of best fit” of the full load/displacement (stress/strain) curve or a portion of the load/displacement (stress/strain) curve over a truncated range of displacements (e.g., from 0.5 inches to 1.0 inches). In this way, the tightness of the tissue is a measure of the load required to displace the tissue per unit of displacement. The load/displacement (stress/strain) curve or the calculated tissue tightness may then be displayed to the user via the display on the data acquisition unit 103 or electrosurgical generator unit 50. The user could then use this information to determine the extent of treatment achieved and make decisions on whether additional treatment is needed to achieve a pre-determined level of tissue tightening.

It is to be appreciated that the suction cup 108 may cause slight bruising on the tissue depending on the amount of suction pressure exerted on the tissue. To reduce or eliminate bruising, a thin, adhesive backed film or protective patch may be placed on the tissue prior to application of the suction cup 108 to tissue. This film or protective patch provides the additional benefit of marking the location of the measurement taken so that subsequent measurements can be taken in the same location to facilitate comparison between measurements at different points in the treatment. The thickness of the film or protective patch must be thick enough to provide protection against bruising but thin enough to not alter the mechanical properties of the tissue being measured.

Although only one position sensor 140 may be employed, it is to be appreciated that at least two position sensors 140 may be employed to compensate for the uneven or non-uniform nature of tissue or skin. In one embodiment, the at least two position sensors 140 may be averaged to determine a single displacement value. In another embodiment, if the measured difference between the at least two sensors 140 is greater than a predetermined threshold, an error may be generated to alert the user. The error and/or alert may be transmitted to the data acquisition unit 103 where the error or alert may be presented to the user on a display or via an annunciator.

In a further embodiment, three position sensors may be employed to further average out inconsistencies. Referring to FIGS. 13A-13C, a tissue tightness measuring device 201 employing three displacement or position sensors are provided. It is to be appreciated that device 201 functions substantially in the same manner as described above for device 101. In this embodiment, the controller disposed in the housing 202 may average measured displacement from the three sensors to determine a single displacement value.

It is to be appreciated that the position sensors are appropriately spaced to measure a relatively flat surface and avoid measuring “tenting skin”. As shown in FIG. 13C, the position sensors 240 are positioned close to the circumference 209 of the lower housing 206 of device 201 to avoid tenting skin. As shown in FIG. 13A, the alignment devices 242 cause alignment indicators 262 to appear on the tissue 230. The alignment indicators 230 indicate where the position sensors 240 are taking a measurement. As can be seen in FIG. 13A, the position sensor reading areas (as indicated by inverted triangle 265) are outside the area of the tenting tissue 231.

In a further embodiment, the position sensors 240 may be adjustable and may move in a radial direction from the circumference 209 of the lower housing 206 toward the center of the lower housing 209 and vice versa. In this manner, the sensors 240 may be positioned closer to the center of the lower housing 206 when taking measurements on a narrow part of the body and the sensors 240 may be positioned closer to the circumference 209 of the lower housing 206 when taking measurements where a large amount of tenting occurs.

It is to be appreciated that the tissue tightness measurement devices 101, 201 of the present disclosure may be used to provide tissue tightness measurements or other relevant information (e.g., tissue displacements, forces, etc.) to generator 50. Generator 50 may be coupled to a display device (or optionally, the generator may have a display device integrated into the generator housing) and may output for display the current tissue tightness, a stress-strain curve, or any other relevant data. In one embodiment, if a processor in generator 50 determines that the slope of a stress-strain curve is constant (e.g., the tissue tightness is no longer changing), generator 50 outputs an alert or notification indicating that the skin surface and/or tissue plane of interest should no longer be displaced to prevent damage or permanent deformation of the patient tissue. In other embodiment, the generator 50 may output an alert or indication that a level of tissue tightness has been achieved, for example, a tissue tightness measurement is greater than a baseline measurement, a tissue tightness measurement is greater than a predetermined tissue tightness value, etc.

It is to be appreciated that the terms “tissue plane”, “tissue plane of interest”, “subdermal tissue plane”, “subcutaneous tissue plane”, as used herein includes all layers or planes of patient tissue below the tissue surface or dermis of the patient and until the muscle.

It is to be further appreciated that alternative embodiments to the displacement mechanism such as suction cup 108 may exist for attaching the tissue to be measured to the load cell/sensor 110. In one embodiment, an interface may be provided and configured to be attached to or grip the patient tissue (e.g., a skin surface) and also be attached to the load cell/sensor 110. For example, in one embodiment, the interface may be suture loops placed through the dermis/skin, where the suture loops may be attached to the lower arm 112 of the load cell/sensor 110. In another embodiment, the interface may include a pad having an adhesive backing and a hook, loop, or other means for enabling the adhesive backed pad to be attached to the load cell/sensor 110. Alternatively, glue may be used to grip a side of the pad to the skin surface. For example, a pad having adhesive on both sides may be employed to couple the pad to the tissue of interest, e.g., skin, and the load cell/sensor 110. In another embodiment, an adhesive backed pad may be used for gripping the skin surface and suture loops may be threaded through both the pad and the dermis/skin, where the loops may then be attached to the load cell/sensor 110. It is to be appreciated that the adhesive backed pad may be configured in a similar manner to ECG pads, steri-strips, or other adhesively backed pads. It is further to be appreciated that when using alternative embodiments to the displacement mechanism (i.e., not using suction cup 108), button 133 may be employed to initiate/stop acquisition of data in lieu of employing switch 128.

It is to be appreciated that the various features shown and described are interchangeable, that is a feature shown in one embodiment may be incorporated into another embodiment.

While the disclosure has been shown and described with reference to certain preferred embodiments thereof, it will be understood by those skilled in the art that various changes in form and detail may be made therein without departing from the spirit and scope of the disclosure as defined by the appended claims.

Furthermore, although the foregoing text sets forth a detailed description of numerous embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean...” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

What is claimed is:

1. A tissue tightness measurement device comprising:
a displacement mechanism that displaces tissue;
at least one position sensor that measures displacement of the tissue;
a load sensor that measures force applied to the tissue as the tissue is being displaced, the load sensor being coupled to the displacement mechanism; and
a controller configured to determine a value of the tightness of the tissue based on the measured displacement of the tissue and the measured force applied to the tissue during the displacement.

2. The device of claim 1, wherein the controller generates a force/displacement curve based on the measured displacement of the tissue and the measured force applied to the tissue during the displacement.

3. The device of claim 2, wherein the controller determines the value of the tightness of the tissue by calculating a slope of the line of best fit over at least a portion of the force/displacement curve for a range of displacements.

4. The device of claim 1, wherein the displacement mechanism is a suction cup that draws tissue into the suction cup when a vacuum is applied to the suction cup.

5. The device of claim 4, further comprising a switch disposed in the suction cup and coupled to the controller, wherein when tissue is drawn into the suction cup and makes contact with the switch, the switch sends a signal to the controller to initiate acquisition of measurements.

6. The device of claim 5, wherein when tissue overcomes the vacuum and disengages from the switch, the switch sends a signal to the controller to stop acquisition of measurements.

7. The device of claim 1, wherein the displacement mechanism is an interface including suture loops placed through the tissue and coupled to the load sensor.

8. The device of claim 1, wherein the displacement mechanism is an interface including an adhesive pad that is coupled to the tissue and to the load sensor.

9. The device of claim 1, wherein the at least one position sensor includes a transmitter and receiver to determine a distance from the at least one position sensor to a surface of the tissue.

10. The device of claim 9, wherein at least two position sensors are employed to compensate for non-uniform tissue.

11. The device of claim 9, wherein at least two position sensors are employed to compensate for tilting of the device by the user during measurements.

12. The device of claim 1, further comprising at least one alignment device to align the at least one position sensor to a desired location on tissue.

13. The device of claim 12, wherein two alignment devices are positioned on opposite sides of each of the at least one position sensor.

14. The device of claim 1, further comprising a transceiver that sends and receives data and/or signals with an external device.

15. The device of claim 1, further comprising a housing, wherein the at least one position sensor is adjustably disposed on a surface of the housing.

16. The device of claim 15, wherein the housing is generally cylindrical and the at least one position sensor is radially adjustable to avoid taking measurements on tenting tissue.

17. The device of claim 1, wherein the controller is coupled to a data acquisition and display device that displays at least one of measurements taken, load/displacement curves and/or values of tissue tightness.

18. The device of claim 17, wherein the controller communicates to the data acquisition and display device via hardwired or/and wireless means.

19. The device of claim 17, wherein the data acquisition and display device is an electrosurgical generator unit (ESU).

20. The device of claim 19, wherein the ESU uses data provided from the controller to control a tissue tightening procedure and/or to determine the effectiveness of the tissue tightening procedure.

21. A system comprising:
an electrosurgical generator unit (ESU) that generates electrosurgical energy;
an applicator configured to alter tissue, the applicator being configured to receive the electrosurgical energy from the ESU to energize an electrode disposed in the applicator and to generate plasma when an inert gas is passed over the energized electrode, wherein the plasma alters the tissue; and
a tissue tightness measurement device that determines tightness of the altered tissue.

22. The system of claim 21, wherein the tissue tightness measurement device comprises:
a displacement mechanism that displaces tissue;
at least one position sensor that measures displacement of the tissue;
a load sensor that measures force applied to the tissue as the tissue is being displaced, the load sensor being coupled to the displacement mechanism; and
a controller configured to determine a value of the tightness of the tissue based on the measured displacement of the tissue and the measured force applied to the tissue during the displacement.

23. The system of claim 22, wherein the controller generates a force/displacement curve based on the measured displacement of the tissue and the measured force applied to the tissue during the displacement.

24. The system of claim 23, wherein the controller determines the value of the tightness of the tissue by calculating a slope of the line of best fit over at least a portion of the force/displacement curve for a range of displacements.

25. The system of claim 22, wherein the ESU further comprises a display device that displays at least one of measurements taken, load/displacement curves and/or values of tissue tightness.

26. The system of claim 22, wherein the controller communicates to the ESU via hardwired or/and wireless means.

27. The system of claim 22, wherein the ESU uses data provided from the controller of the tissue tightness measurement device to control a tissue tightening procedure and/or to determine the effectiveness of the tissue tightening procedure.

28. The system of claim 22, wherein the displacement mechanism is a suction cup that draws tissue into the suction cup when a vacuum is applied to the suction cup.

29. The system of claim 28, wherein the tissue tightness measurement device further comprising a switch disposed in the suction cup and coupled to the controller, wherein when tissue is drawn into the suction cup and makes contact with the switch, the switch sends a signal to the controller to initiate acquisition of measurements.

30. The system of claim 29, wherein when tissue overcomes the vacuum and disengages from the switch, the switch sends a signal to the controller to stop acquisition of measurements.
